SEQUENCE LISTING (1 sequence)

SEQ ID NO: 1 - DNA, 930 bp, Bacillus altitudinis
tacggtgcgtgctagtcgacggcagcggttataaagtatctggcggtctgcatggtgtaggggcatctgttgttaatgcgttatctacgaccttagacgtgaccgtataccgtgatggaaaaattcattaccagcaattcaaacgcggcgttccagttggagatttagaggtcattggtgaaacagatgtaacagggacaacgactcattttgtgccagatccagaaattttcactgaaaccattgaatttgattacgacacacttgctaaccgtgtacgtgagttagctttcttaacaaaaggcgtcaacatcatcattgaagacttacgtgaaggcaaagagcgaagaaacgaatactgctatgaaggcggtattaagagctatgtagaacatttgaatcgctcgaaggaagtcgttcatgaagaaccagtttacatcgagggtgaaaaagacggaatcaccgttgaagtggcactgcaatacaacgattcctatacaagcaatatttattctttcgccaacaacatcaacacatatgaaggcggaacacacgaagctggctttaaaaccggtctgacgcgtgtcatcaatgattatgctcgtaaaaatggcgtattcaaagatggagactcgaatttgagcggtgaagatgtacgagaaggcttaacagccattatctctatcaaacatccagaccctcaattcgaaggacaaacgaagacaaagctcggtaactcagaagcaagaaccattaccgactccctcttctccgaagcacttgagaaattcctcttagagaaccctgatgctgcaaagaaaattgtggagaaaggtgtgatggcagctcgtgcaagaatggcctgccaaaaaaggcacgtgagctgacaagacgtaaaagtgcactggaagtctctagcttgcctgcaaa

(19) United States
(12) Patent Application Publication
(10) Pub. No.: US 2023/0059898 A1
(43) Pub. Date: Feb. 23, 2023

(54) BACILLUS ALTITUDINIS ANTAGONIZING XANTHOMONAS ORYZAE AND USE THEREOF

(71) Applicant: JIANGSU ACADEMY OF AGRICULTURAL SCIENCES, Nanjing, Jiangsu (CN)

(72) Inventors: Fengquan LIU, Nanjing (CN); Yancun ZHAO, Nanjing (CN); Weibo SUN, Nanjing (CN); Chengqi MIAO, Nanjing (CN); Chaohui LI, Nanjing (CN); Bao TANG, Nanjing (CN); Xian CHEN, Nanjing (CN); Gaoge XU, Nanjing (CN); Yangyang ZHAO, Nanjing (CN); Liang MING, Nanjing (CN); Baodian GUO, Nanjing (CN)

(73) Assignee: JIANGSU ACADEMY OF AGRICULTURAL SCIENCES, Nanjing, Jiangsu (CN)

(21) Appl. No.: 17/785,529

(22) PCT Filed: Aug. 11, 2021
(86) PCT No.: PCT/CN2021/112081

§ 371 Date: Jun. 15, 2022

(30) Foreign Application Priority Data
Oct. 20, 2020 (CN) 202011124578.X

(51) Int. Cl. A01N 63/22; C12N 1/20; A01P 15/00

(52) U.S. Cl. CPC A01N 63/22; C12N 1/205; A01P 15/00; C12R 2001/07

(57) ABSTRACT

A Bacillus altitudinis ST15 antagonizing Xanthomonas oryzae and use thereof, the Bacillus altitudinis ST15 was deposited in China General Microbiological Culture Collection Center on Jun. 28, 2020 and has the deposit number of CGMCC No. 20156. The Bacillus altitudinis has a relatively high antagonistic activity to Xanthomonas oryzae pv. oryzae and Xanthomonas oryzae pv. oryzicola, can promote growth of rice and improve a drought resistance of a rice seedling, and has a relatively strong tolerance to common chemical microbicides for controlling bacterial diseases of rice. A bacterial suspension or a biocontrol inoculant prepared from this strain has a relatively high biocontrol effect on bacterial blight of rice and bacterial leaf streak of rice, can replace or reduce chemical pesticides, improves safety of food and ecological environment, and has relatively high economic and social benefits.

### TECHNICAL FIELD

The present invention belongs to the technical field of biology, and particularly relates to a Bacillus altitudinis ST15 antagonizing Xanthomonas oryzae and use thereof.

### BACKGROUND

Xanthomonas oryzae comprises two pathogenic varieties of Xanthomonas oryzae pv. oryzae and Xanthomonas oryzae pv. oryzicola respectively causing bacterial blight (BB) of rice and bacterial leaf streak (BLS) of rice. The two bacterial diseases mainly occur in rice production in China, have an annual occurrence area of 6.67×10⁵-1×10⁶ hectares and a damage area of 3.33×10⁵-5.33×10⁵ hectares, and generally causes the yield loss of 5-10%, seriously 50% or more and even a total failure. In recent years, due to popularization of a simplified planting mode of rice, a frequent exchange of germplasm resource, a pathogenic variation of pathogenic bacteria and the like, the two bacterial diseases are not only continuously epidemic in an indica rice region in south China, but also quickly spread to a mixed planting region of indica rice and japonica rice in Yangtze River basin and a japonica rice region in north China, and thus seriously threatening safe production of rice in China. At present, chemical pesticides are mainly used for controlling the bacterial diseases of rice, including trichloroisocyanuric acid, chlorobromoisocyanuric acid, thiediazole copper, benziothiazolinone and zinc thiazole. However, long-term unreasonable use of the chemical pesticides leads to a gradual enhancement of a pesticide resistance of pathogenic bacteria, a blind increase of an application dosage and an unsatisfactory control effect. The chemical pesticides have a comprehensive control effect of 50-70% on diseases. Meanwhile, residual pesticides cause a severe threat to food safety and ecological environment. In addition, due to a high cost and a great investment risk in developing safe and efficient chemical microbicides, novel chemical microbicides for controlling crop bacterial diseases are rare in recent years.

Biopesticides have increasingly become a key point for developing international green pesticides due to advantages including relative safety, no residue and environmental friendliness. A microbial-based biocontrol technology is considered to be one of the major development directions for controlling the crop bacterial diseases in the future. Bacillus spp. belongs to gram-positive bacteria, is widely distributed in various habitats including soil, plant surfaces, water bodies and agricultural wastes, for example, Bacillus amyloliquefaciens (B. amyloliquefaciens), Bacillus subtilis (B. subtilis), Bacillus polymyxa (B. polymyxa), Bacillus firmus (B. firmus) and Bacillus cereus (B. cereus), and the Bacillus spp. has a broad-spectrum antibacterial activity, a high spore yield and a strong stress adaptability, and is considered to be one of most beneficial microorganisms with use and development potentials and a biocontrol microorganism widely accepted by scientists and consumers and safe to human bodies. Currently, there are 137 bactericide products containing Bacillus spp. active ingredients registered in China.

Compared with the Bacillus spp., there are rare research reports on controlling crop diseases by Bacillus altitudinis. Different-derived strains have significant different biological characteristics and antibacterial spectrum, and have specificity. The invention patent CN105820981A discloses that a Bacillus altitudinis strain has a good biocontrol effect on various fungal diseases such as peach brown rot, gray mold, soft rot and penicilliosis. The patent CN108865946A discloses that a Bacillus altitudinis strain has a good control effect on tomato root knot nematode. It is of great significance in studying a strain having a control effect on bacterial blight of rice and bacterial leaf streak of rice.

### SUMMARY

The present invention aims to provide a Bacillus altitudinis ST15 having a significant control effect on bacterial blight of rice and bacterial leaf streak of rice and use thereof. The Bacillus altitudinis strain of the present invention has a high growth speed, a strong antagonistic activity, a good biocontrol effect and a strong tolerance to chemical microbicides, and can promote growth of rice and improve a drought resistance of rice.

In order to solve the above problems, the technical solutions adopted by the present invention are as follows:

A biocontrol strain ST15 that has a strong antagonistic activity against bacterial blight of rice and bacterial leaf streak of rice is separated and screened from the rhizosphere soil of a pear orchard by using a dilution coating separation method and a plate antagonism method. The strain is identified as Bacillus altitudinis by combination of colony morphology observation, bacterial morphology observation, biochemical indicators and gyrB gene sequence determination. The strain was deposited in China General Microbiological Culture Collection Center (CGMCC) located at No. 3, Yard 1, West Beichen Road, Chaoyang District, Beijing, Institute of Microbiology, Chinese Academy of Sciences (postcode: 100101) on Jun. 28, 2020 and has the deposit number of CGMCC No. 20156.

The Bacillus altitudinis strain ST15 is cultured on an NA solid medium plate at 28° C. for 48 h. After the culture, a single colony presents milky white, flat, round or nearly round, has a diameter of 2.0 mm-4.0 mm, and has a shrunken surface and a neat edge; and the strain is rod-shaped, has a diameter of about 0.5 μm-0.6 μm and a length of about 1.5 μm-2.0 μm and fusiform spores. The NA solid medium contains 5 g of peptone, 10 g of sucrose, 1 g of a yeast extract, 3 g of a beef extract and 15 g of agar powder, water is added to a constant volume of 1,000 mL, pH is adjusted to 6.8-7.2, and the medium is sterilized at 121° C. for 20 min.

An indoor NA solid medium plate bioassay is used to determine tolerance of Bacillus altitudinis ST15 to zinc thiazole, benziothiazolinone and trichloroisocyanuric acid.

The present invention provides a fermentation product of the Bacillus altitudinis ST15. The fermentation product of the present invention may be in any conventionally fermented product form in the art, for example, including but not limited to a fermentation broth containing bacteria, or a sterile fermentation filtrate after bacteria removing by filtration or an effective ingredient obtained by extraction after fermentation of the strain.

In some embodiments of the present invention, a fermentation broth of the Bacillus altitudinis ST15 provided by the present invention may refer to a fermentation broth containing bacteria; and in other embodiments of the present invention, a sterile fermentation filtrate of the Bacillus altitudinis ST15 may refer to a liquid after the fermentation broth is filtered to remove the bacteria.

The present invention also provides a bacterial suspension containing the Bacillus altitudinis ST15, which can be a solution obtained by resuspending bacteria obtained by centrifugation according to conventional methods and conventional reagents in the art.

The present invention further provides a biocontrol inoculant containing the Bacillus altitudinis ST15 or a fermentation product of the Bacillus altitudinis ST15.

The fermentation product, the fermentation broth, the sterile fermentation filtrate, and the biocontrol inoculant of the present invention can be prepared according to conventional methods in the art. For example, after a Bacillus altitudinis ST15 is prepared into a seed broth, the seed broth is inoculated into a fermentation medium to obtain the fermentation broth; after the fermentation broth is centrifuged and filtered, the sterile fermentation filtrate is obtained; and the biocontrol inoculant is obtained by adding auxiliary agents permitted in the art into the fermentation broth.

In a specific embodiment, the present invention provides a method for preparing a biocontrol inoculant containing the Bacillus altitudinis ST15 and/or a fermentation product of the Bacillus altitudinis ST15:

(1) Activation culture: the Bacillus altitudinis ST15 of the present invention preserved in glycerol is taken, the bacteria are inoculated on a fresh NA solid plate by streaking, and the bacteria is cultured at 26° C.-30° C. for 24 h-36 h;

(2) Seed broth preparation: a single colony is picked and the colony is inoculated in an NB liquid medium, and the bacteria is cultured at 26° C.-30° C. for 16 h-24 h under oscillation at an oscillation frequency of 100 r/min-200 r/min;

(3) Preparation of biocontrol bacteria liquid: the seed broth obtained in step (2) is inoculated into a 40%-60% NB medium at a ratio of 1%-5% (v/v) for fermentation at a temperature of 27° C.-30° C., a loading volume of 200-400 mL/1,000 mL, an initial pH of the medium of 6.8-7.2 and a stirring speed of 100 r/min-180 r/min for 48 h-72 h; and bacterial density of a fermentation broth is more than 1.95×10⁹ cfu/mL counted by a plate coating method;

(4) Preparation of a Bacillus altitudinis suspension: an obtained ST15 fermentation broth prepared in step (3) is centrifuged for 10 min-15 min, a supernatant is removed, the bacteria are collected and suspended in sterile water, the bacterial density is adjusted to 1.0×10⁷-1.0×10⁹ cfu/mL, and Tween 80 is added at 0.05%-0.15% (v/v) of the total volume;

(5) Preparation of a fermentation broth filtrate: an obtained ST15 fermentation broth prepared in step (3) is centrifuged for 10 min-15 min, and a supernatant is taken and filtered with a bacterial filter to obtain a sterile fermentation filtrate; and

(6) Preparation of a biocontrol inoculant: conventional auxiliary agents in the art are added to a fermentation broth prepared in step (3), a pH value is adjusted to 4-5, and an obtained biocontrol inoculant is aseptically filled and stored at a room temperature.

The conventional auxiliary agents in the preparation of the biocontrol inoculant of the present invention can be selected according to a conventional method in the art. For example, in a specific embodiment of the present invention, a surfactant fatty alcohol-polyoxyethylene ether, a thickener xanthan gum, an antifoaming agent polydimethylsiloxane and a preservative sodium benzoate are added. In some more specific embodiments of the present invention, in the preparation of the biocontrol inoculant, the auxiliary agents contain the following components in the mass percentages: 1.0%-2.0% of fatty alcohol-polyoxyethylene ether, 0.2%-0.3% of xanthan gum, 0.2%-0.5% of polydimethylsiloxane and 0.1%-0.15% of sodium benzoate. Here is only one recited embodiment, but it is not limited to the method of adding the auxiliary agents, and other solutions capable of forming an inoculant dosage form are acceptable.

In some embodiments of the present invention, a 50% NB medium contains 2.5 g of peptone, 5 g of sucrose, 0.5 g of a yeast extract and 1.5 g of a beef extract, water is added to a constant volume of 1,000 mL, pH is adjusted to 6.8-7.2, and the medium is sterilized at 121° C. for 20 min.

In some embodiments of the present invention, an NA solid medium contains 5 g of peptone, 10 g of sucrose, 1 g of a yeast extract, 3 g of a beef extract and 15 g of agar powder, water is added to a constant volume of 1,000 mL, pH is adjusted to 6.8-7.2, and the medium is sterilized at 121° C. for 20 min.

A sterile supernatant of the Bacillus altitudinis ST15 fermentation broth of the present invention has high tolerance to a high temperature, ultraviolet rays, a trypsin, a proteinase K, a pepsin, and an acid-base environment.

The present invention also provides use of the Bacillus altitudinis ST15 or the fermentation product thereof in soaking a rice seed and thus promoting rice growth or/and improving a drought resistance of a rice seedling.

The present invention also provides use of the Bacillus altitudinis ST15, the bacterial suspension, the fermentation product, the fermentation broth or the sterile fermentation filtrate in promoting growth of rice or/and improving a drought resistance of a rice seedling.

In one embodiment, the use of the present invention in promoting growth of rice is specifically as follows: using the Bacillus altitudinis ST15 of the present invention, the bacterial suspension, the fermentation product, the fermentation broth or the sterile fermentation filtrate to treat a rice seed at 25° C.-30° C. for 36 h-48 h.

In one embodiment, the use of the present invention in improving a drought resistance of a rice seedling is specifically as follows: using the Bacillus altitudinis ST15 of the present invention, the bacterial suspension, the fermentation product, the fermentation broth or the sterile fermentation filtrate to treat a rice seed at 25° C.-30° C. for 36 h-48 h.

For the present invention, the rice seed is treated by soaking, and in some embodiments, a liquid containing the Bacillus altitudinis ST15 may be at a bacterial density of 1.0×10⁷-1.0×10⁹ cfu/mL.

The present invention also provides use of the Bacillus altitudinis ST15, the fermentation product, the fermentation broth or the sterile fermentation filtrate, the bacterial suspension or the biocontrol inoculant in controlling bacterial blight of rice and/or bacterial leaf streak of rice.

In one embodiment, specifically the use of the present invention in controlling the bacterial blight of rice is as follows: spraying before rice is infected with Xanthomonas oryzae pv. oryzae or after rice is infected with Xanthomonas oryzae pv. oryzae.

In one embodiment, specifically the use of the present invention in controlling the bacterial leaf streak of rice is as follows: spraying before rice is infected with Xanthomonas oryzae pv. oryzicola or at an initial stage of the infection and spraying 1-2 times at an interval of 7-10 d.

In some specific embodiments, a liquid used in the spraying contains Bacillus altitudinis ST15 at a bacterial density of 1.0×10⁷-1.0×10⁹ cfu/mL.

Compared with the related art, the present invention has the following advantages: The Bacillus altitudinis ST15 of the present invention is a newly found Bacillus spp. biocontrol resource, has a relatively high antagonistic activity to Xanthomonas oryzae pv. oryzae and Xanthomonas oryzae pv. oryzicola, and presents relatively strong tolerance to chemical microbicides for controlling bacterial diseases of rice. A bacterial suspension prepared from the present invention is used to treat a rice seed and thus can promote growth of rice and improve a drought resistance of a rice seedling. A biocontrol inoculant prepared from the strain of the present invention has a relatively high biocontrol effect on bacterial blight of rice and bacterial leaf streak of rice, can replace or reduce chemical pesticides, improves safety of food and ecological environment, and has relatively high economic and social benefits.

### BRIEF DESCRIPTION OF THE DRAWINGS

FIG. 1 shows a plate antagonistic activity of 1 preliminarily identified biocontrol bacterial strain against Xanthomonas oryzae pv. oryzae and Xanthomonas oryzae pv. oryzicola;

FIG. 2 shows a diameter of an antagonistic circle of 1 preliminarily identified biocontrol bacterial strain against Xanthomonas oryzae pv. oryzae and Xanthomonas oryzae pv. oryzicola;

FIG. 3 shows morphologies of colonies, bacteria and spores of a Bacillus altitudinis ST15 on an NA medium plate;

FIG. 4 shows a phylogenetic tree analysis based on a gyrB gene sequence of a Bacillus altitudinis ST15;

FIG. 5 shows growth dynamics of a Bacillus altitudinis ST15 in an NB medium;

FIG. 6 shows an antibacterial activity and a stability analysis of a supernatant of a fermentation broth of a Bacillus altitudinis ST15;

FIG. 7 shows a tolerance of a Bacillus altitudinis ST15 to 3 chemical microbicides;

FIG. 8 shows an effect of a Bacillus altitudinis ST15 on promoting growth of rice;

FIG. 9 shows a statistical analysis of a Bacillus altitudinis ST15 on promoting growth of buds and roots of rice;

FIG. 10 shows that a Bacillus altitudinis ST15 significantly improves a drought resistance of a rice seedling;

FIG. 11 shows an inhibitory effect of a Bacillus altitudinis ST15 bacterial suspension on spot expansion of bacterial blight of rice; and

FIG. 12 shows an effect of a Bacillus altitudinis ST15 bacterial suspension on controlling bacterial blight of rice.

### DETAILED DESCRIPTION

The Bacillus altitudinis strain ST15 of the present invention was deposited in China General Microbiological Culture Collection Center (CGMCC) located at No. 3, Yard 1, West Beichen Road, Chaoyang District, Beijing, Institute of Microbiology, Chinese Academy of Sciences (postcode: 100101) on Jun. 28, 2020 and has the deposit number of CGMCC No. 20156. Xanthomonas oryzae pv. oryzae strains YN1, YN24, GD414 and PX099, and Xanthomonas oryzae pv. oryzicola strains R5105, Xoc197, Xoc-S and Xoc-M used in the present invention are all preserved in this laboratory, and other units or individuals can obtain the above 8 pathogenic bacterial strains from this laboratory.

The following embodiments are used to illustrate the present invention, but do not limit the present invention. If there are no special instructions, the technical methods used in the following embodiments are all conventional; and if there are no special instructions, the experimental materials used in the following embodiments are all conventional chemical reagents and biochemical reagents.

The NB medium contains 5 g of peptone, 10 g of sucrose, 1 g of a yeast extract and 3 g of a beef extract, water is added to a constant volume of 1,000 mL, pH is adjusted to 6.8-7.2, and the medium is sterilized at 121° C. for 20 min; and an NA solid medium is obtained by adding 15 g/L of agar powder into the NB medium.

The 50% NB medium contains 2.5 g of peptone, 5 g of sucrose, 0.5 g of a yeast extract and 1.5 g of a beef extract, water is added to a constant volume of 1,000 mL, pH is adjusted to 6.8-7.2, and the medium is sterilized at 121° C. for 20 min.

The following embodiments are only part of the preferred embodiments of the present invention and are used only to describe the present invention and are not to be construed as limiting the scope of the present invention. It should be noted that any modification, equivalent replacement, or improvement made and the like within the spirit and principle of the present invention shall fall within the protection scope of the present invention.

### Embodiment 1

Isolation and Identification of Bacillus altitudinis Strain ST15

(1) Separation of strain resources to be screened: 50 g of soil was randomly taken from the rhizosphere of a pear orchard, a total of 10 soil samples were taken to be mixed evenly, 10 g of soil was put into a 250 mL conical flask containing 20 fine glass beads and 100 mL of sterilized deionized water, the flask was placed on a shaker at 150 r/min and a shake culture was conducted at 28° C. for 1 h. The cultured strain was put still for 10 min, 1 mL of a supernatant was taken and diluted in a 10-fold gradient manner by using sterilized deionized water, 100 μL of a diluted solution of each gradient concentration was coated on an NA solid plate, and a static culture was conducted at 28° C. for 36 h. Single colonies were picked by using an inoculating loop to streak on the NA solid medium for purification 2 times, the purified single colonies of different forms were transferred to an NA solid plate, an inverted culture was conducted at 28° C. for 48 h, and the strains were stored at 4° C. for later use.

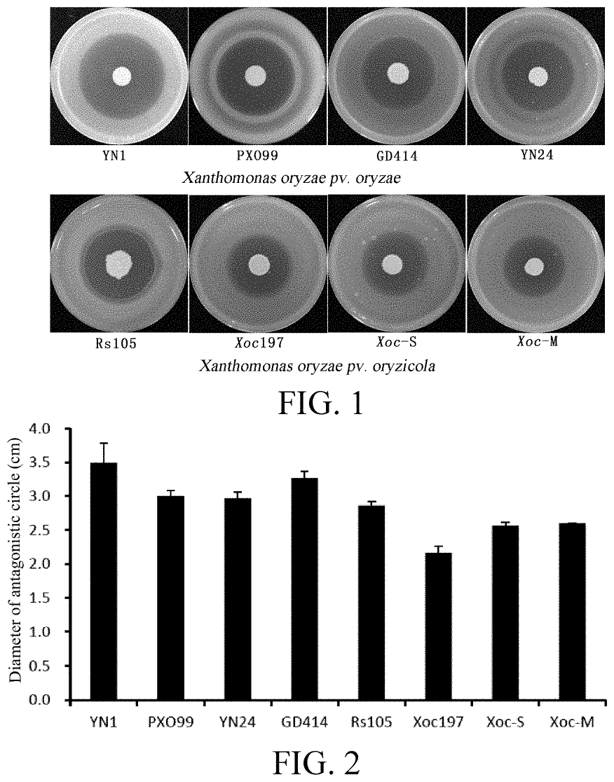

(2) Plate antagonistic screening of biocontrol strains: each purified strain stored on the NA solid plate was cultured in an NB liquid medium at 150 r/min and 28° C. for 36 h as a seed broth for later use; Xanthomonas oryzae pv. oryzae YN1 and Xanthomonas oryzae pv. oryzicola Rs105 preserved in glycerol were separately streaked onto the NA solid plate and cultured at 28° C. for 48 h, single colonies were picked and inoculated into a 250 mL conical flask containing 50 mL of the NB liquid medium and cultured at 150 r/min and 28° C. for 36 h, and a culture medium (OD₆₀₀≈1.0) was added into a liquid low-temperature NA agar medium to be mixed evenly quickly to prepare an NA solid nutritional plate; 2 μL of the seed broth of the strains to be tested was inoculated in droplets on the prepared NA solid nutritional plate; a treated antagonistic plate was cultured at 28° C. for 48 h, and an antagonistic activity of the strains to be tested on the Xanthomonas oryzae pv. oryzae YN1 and the Xanthomonas oryzae pv. oryzicola Rs105 was evaluated by a diameter of an antagonistic circle; and the strains with the diameter of the antagonistic circle larger than 2.0 cm were then selected and an antagonistic activity of the primarily screened antagonistic strains on Xanthomonas oryzae pv. oryzae YN24, PX099 and GD414, and Xanthomonas oryzae pv. oryzicola Xoc197, Xoc-S and Xoc-M with different pathogenicity was determined according to the method. The results showed that among the tested 652 strains, the strain numbered ST15 had a best performance. The ST15 strain had an antagonistic circle diameter of 2.97 cm-3.50 cm against 4 strains of Xanthomonas oryzae pv. oryzae and an antagonistic circle diameter of 2.17 cm-2.87 cm against 4 strains of Xanthomonas oryzae pv. oryzicola as shown in FIG. 1 and FIG. 2. Each strain was subjected to the antagonistic determination in 3 replicates.

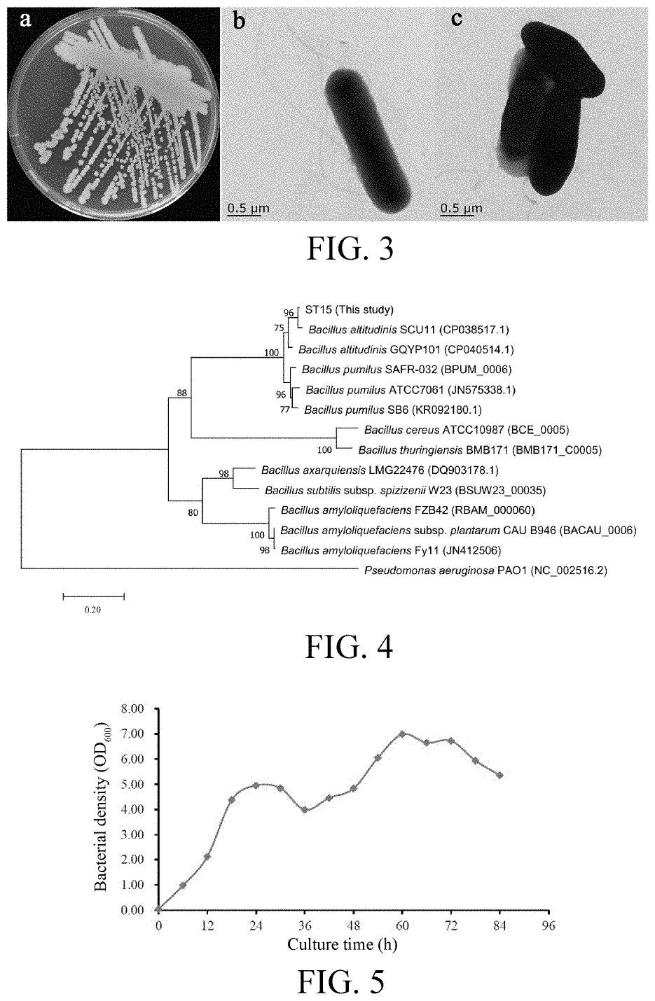

(3) Observation of morphology of biocontrol strain ST15: bacteria were picked from an ST15 plate stored at 4° C. by using a sterile inoculating loop and streaked to be cultured on an NA solid plate at 28° C. for 48 h; a single colony presented milky white, flat, round or nearly round, had a diameter of 2.0 mm-4.0 mm, and had a shrunken surface and a neat edge; the bacteria cultured for 24 h were picked and observed by using a transmission electron microscope, and the strain was rod-shaped, had a diameter of about 0.5 μm-0.6 μm and a length of about 1.5 μm-2.0 μm; and a culture after 48 h was picked from the NA solid plate and observed by using the transmission electron microscope, most bacteria were transformed into spores and the spores were fusiform and had a diameter of about 0.5 μm-0.6 μm and a length about 1.0 μm-1.5 μm as shown in FIG. 3.

(4) Physical and chemical experiment analysis of biocontrol strain ST15

Results of a physical and chemical experiment analysis of a biocontrol strain ST15 were as shown in Table 1.

TABLE 1
Results of physical and chemical experiment analysis of biocontrol strain ST15

| | | Experiment | | Experiment | | Experiment | |
|---|---|---|---|---|---|---|---|
| Experiment Item | Result | Item | Result | Item | Result | Item | Result |
| Gram staining | Positive | Contact | − | Oxidase | − | Formation of spores | + |
| | | enzyme | | | | | |

BIOLOG GEN III (growth experiment)

| | | | | | | | |
|---|---|---|---|---|---|---|---|
| Negative control | − | α-D-glucose | + | Gelatin | + | α-D-lactose | − |
| 3-methyl-D-glucose | − | L-fucose | + | Gentiobiose | + | D-melibiose | + |
| D-glucose-6-phosphate | − | D-fructose | + | L-alanine | + | L-lactic acid | − |
| Glycine-L-proline | + | D-galactose | + | L-arginine | + | Citric acid | + |
| p-hydroxyphenylacetic acid | − | Dextrin | − | L-aspartic | + | D-malic acid | − |
| | | | | acid | | | |
| Methyl pyruvate | − | Inosine | + | L-glutamic | + | L-malic acid | + |
| | | | | acid | | | |
| D-methyl lactate | − | D-sorbitol | − | α- | − | Bromosuccinic acid | + |
| | | | | ketoglutarate | | | |
| β-methyl-D-glucoside | + | D-mannitol | + | D-serine | − | Tween 40 | − |
| β-hydroxy-D,L-butyric acid | − | D-arabitol | − | L-serine | + | α-hydroxybutyric | − |
| | | | | | | acid | |
| Glucuronamide | + | Inositol | − | Pectin | + | D-salicin | − |
| N-acetyl-β-D-mannosamine | + | Glycerol | + | Sucrose | + | α-ketobutyric acid | − |
| N-acetyl-D-galactosamine | − | D-maltose | − | D-glucose | + | Acetoacetic acid | − |
| N-acetylneuraminic acid | − | Quininic acid | + | Galactaric | − | Propionic acid | − |
| | | | | acid | | | |
| γ-aminobutyric acid | + | D-aspartic acid | + | | | | |

BIOLOG GEN III (chemical sensitivity experiment; +, not sensitive; −, sensitive)

| | | | | | | | |
|---|---|---|---|---|---|---|---|
| Positive control | + | Fusidic acid | − | pH 6.0 | + | Nalidixic acid | + |
| Troleandomycin | − | D-serine | − | pH 5.0 | + | Lithium chloride | + |
| Rifamycin SV | − | Lincomycin | + | 1% sodium | + | Dipotassium | + |
| | | | | lactate | | trioxotellurate | |
| Sodium bromate | + | Guanidine | + | 1% NaCl | + | Aztreonam | + |
| | | hydrochloride | | | | | |
| Vancomycin | − | Tetrazolium | − | 4% NaCl | + | Sodium butyrate | + |
| | | violet | | | | | |
| Sodium tetradecyl sulfate | − | Tetrazolium | − | 8% NaCl | + | | |
| | | blue | | | | | |

(5) Molecular identification of biocontrol strain ST15: an ST15 single colony was picked by using a sterilized toothpick into a 250 mL conical flask containing 50 mL of an NB liquid medium and cultured at 150 r/min and 28° C. for 36 h. Total genomic DNA of the ST15 was extracted by using a bacterial genomic DNA extraction kit. A DNA fragment was amplified by PCR with universal primers UP-1

### (5′-GAAGTCATCATGACCGTTCTGCAYGCNGGNGGNAARTTYGA-3′) and UP-2r

(5′-AGCAGGGTACGGATGTGCGAGCCRTCNACRTCNGCRTCNGTCAT-3′) of a bacterial gyrB gene; a PCR product was sequenced by using sequencing primers UP-1S

### (5′-GAAGTCATCATGACCGTTCTGCA-3′) and UP-2Sr

(5′-AGCAGGGTACGGATGTGCGAGCC-3′); and the ST15 gyrB gene had a sequence as shown in SEQ ID NO.1. Based on the obtained gyrB gene sequence and a GenBank database, a phylogenetic tree analysis of the ST15 was performed using MEGA 7.0 software. The strain was aggregated with Bacillus altitudinis as shown in FIG. 4.

Based on the morphologies of the colony, bacteria and spores, a physical and chemical experiment analysis and a molecular identification result, the biocontrol strain ST15 belonged to Bacillus altitudinis.

### Embodiment 2

Growth Dynamics of Bacillus altitudinis ST15 in NB Medium

The ST15 strain stored in glycerol was taken from an ultra-low temperature refrigerator at −70° C., inoculated on an NA solid plate by streaking, and cultured at 28° C. for 36 h; an ST15 single colony was picked by using a sterilized toothpick to be inoculated into a 250 mL conical flask containing 50 mL of an NB liquid medium and cultured at 150 r/min and 28° C. for 36 h as a seed broth; and the seed broth was inoculated at a ratio of 1% (v/v) into a 250 mL conical flask containing 50 mL of the NB liquid medium and cultured at 180 r/min and 28° C. for 84 h, and an absorbance value (OD₆₀₀) of an obtained fermentation broth was measured 1 time every other 6 h after the inoculation. A bacterial density after 60 h of the inoculation was determined and the method was specifically as follows: samples at the above time points were taken and diluted with sterilized water in a 10-fold gradient manner, 100 of a diluted solution of each gradient concentration was drawn and coated on an NA solid plate, and the bacteria were cultured at 28° C. for 48 h and colony counting was conducted; and a spore formation density was investigated after 60 h of the inoculation and the method was specifically as follows: samples at the above time points were taken, subjected to hot-water bath at 80° C. for 30 min and diluted with sterilized water in a 10-fold gradient manner, 100 μL of a diluted solution of each gradient concentration was drawn and coated on an NA solid plate, and the bacteria were cultured at 28° C. for 48 h and colony counting was conducted. The results showed that the OD₆₀₀ absorbance value of the fermentation broth reached a highest value of 6.984 after 60 h of the inoculation as shown in FIG. 5; and the bacterial density reached 3.08×10⁹ cfu/mL after 60 h of the inoculation and the spore density reached 1.31×10⁹ cfu/mL after 60 h of the inoculation. Each treatment was conducted in three replicates.

### Embodiment 3

Antibacterial Activity and Stability Analysis of Supernatant of Fermentation Broth of Bacillus altitudinis ST15

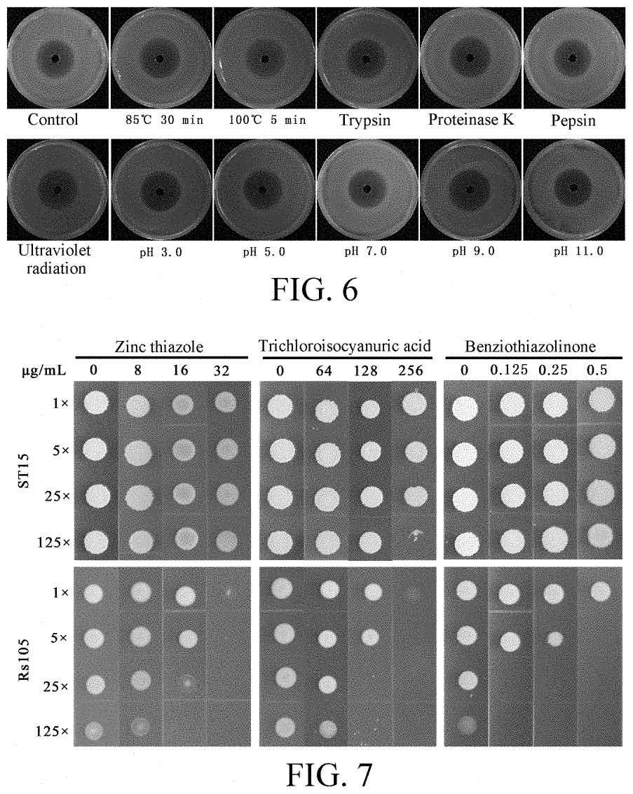

The seed broth of biocontrol bacteria ST15 was prepared according to the method of Embodiment 2, inoculated into a 250 mL conical flask containing 50 mL of a 50% NB medium at a ratio of 2% (v/v) and subjected to a shake culture at 28° C. and 150 r/min. An antibacterial activity of a secondary fermentation metabolite was determined 48 h after the inoculation. The specific method was as follows: samples were collected at the above time point and centrifuged at 10,000 g for 15 min, bacteria were removed by filtration with a 0.22 μm bacterial filter, and 1 mL of a sterile filtrate was sub-packaged into 1.5 mL sterile centrifuge tubes and stored at −70° C. for later use. In order to determine stability of the ST15 secondary antibacterial substance, the sterile filtrates were treated according to the following method, specifically: (1) the sub-packaged sterile filtrates were taken and separately treated in a water bath at 85° C. for 30 min and a water bath at 100° C. for 5 min; (2) the sub-packaged sterile filtrates were separately treated with trypsin, proteinase K and pepsin at a final concentration of 100 μg/mL in a water bath at 37° C. for 6 h; (3) 5 mL of the sterile filtrate was taken and put into a sterile petri dish, a dish lid was opened and the petri dish was placed on an ultra-clean workbench at 60 cm under a 25 W ultraviolet lamp to be irradiated vertically downward for 1 h; and (4) 5 mL of a sterile fermentation broth was put in a 10 mL centrifuge tube, pH was separately adjusted to 3.0, 5.0, 7.0, 9.0 and 11.0, the sterile fermentation broth was placed at a room temperature for 1 h, the pH was adjusted to 7.0, and the sterile fermentation broth was finally filtered with the 0.22 μm bacterial filter. A Rs105 culture medium (OD₆₀₀≈1.0) of Xanthomonas oryzae pv. oryzicola was added to a liquid low-temperature NA medium at a ratio of 1% (v/v) to be mixed evenly quickly to prepare an NA solid nutritional plate; after the plate was solidified, 1 hole with a diameter of 4 mm was made using a sterilized puncher in a center of the plate, 30 μL of the differently treated sterile fermentation filtrate was added into each hole, and the bacteria were cultured at 28° C. for 48 h. The untreated sterile fermentation filtrate was used as a control and each treatment was conducted in 3 replicates. The results showed that under the above different treatments, the sterile fermentation filtrate had a stable antibacterial activity and showed no significant difference from the untreated control as shown in FIG. 6.

### Embodiment 4

Tolerance of Bacillus altitudinis ST15 to 3 Chemical Microbicides

Zinc thiazole, benziothiazolinone and trichloroisocyanuric acid are three main chemical microbicides currently in controlling bacterial blight of rice and bacterial leaf streak of rice in production. In this experiment, a tolerance of a Bacillus altitudinis ST15 to three chemical microbicides was determined indoors, and Xanthomonas oryzae pv. oryzicola Rs105 was used as a control. A specific method was as follows: first, zinc thiazole and benziothiazolinone were respectively dissolved in DMSO, and 20 mg/mL of zinc thiazole mother liquor and 6.4 mg/mL of benziothiazolinone mother liquor were prepared; trichloroisocyanuric acid was dissolved in sterile water and prepared into 7.1 mg/mL of mother liquor; NA solid plates containing zinc thiazole with a final concentration of 0 μg/mL, 8 μg/mL, 16 μg/mL and 32 μg/mL, NA solid plates containing benziothiazolinone with a final concentration of 0 μg/mL, 0.125 μg/mL, 0.25 μg/mL and 0.5 μg/mL, and NA solid plates containing trichloroisocyanuric acid with a final concentration of 0 μg/mL, 64 μg/mL, 128 μg/mL and 256 μg/mL were then separately prepared. A Bacillus altitudinis ST15 culture medium (OD₆₀₀≈0.0) and a Xanthomonas oryzae pv. oryzicola Rs105 culture medium (OD₆₀₀≈0.0) were separately diluted by 5 times, 25 times and 125 times, 2 μL of ST15 and Rs105 stock solutions and the diluted solutions of each concentration were inoculated in droplets on the NA solid plates containing the microbicides, and the bacteria were cultured at 28° C. for 48 h. The results showed that compared with Xanthomonas oryzae pv. oryzicola Rs105, the Bacillus altitudinis ST15 showed stronger tolerance to the three chemical microbicides as shown in FIG. 7. The tolerance of the Bacillus altitudinis ST15 of the present invention to the 3 chemical microbicides indicated that the Bacillus altitudinis can be used with the chemical microbicides in combination.

### Embodiment 5

Experiment of Bacillus altitudinis ST15 on Promoting Growth of Rice

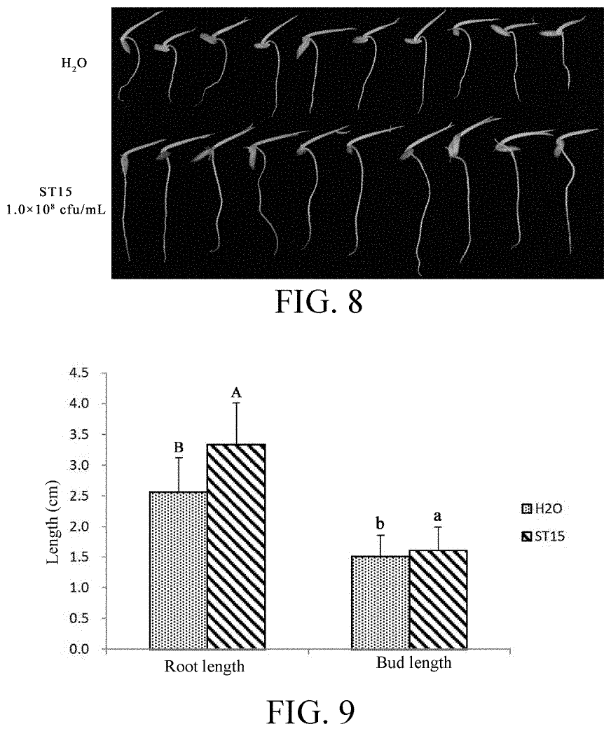

An ST15 seed broth was prepared according to the method of Embodiment 2, inoculated in a 250 mL conical flask containing 50 mL of a 50% NB liquid medium at a ratio of 1% (v/v), and cultured at 180 r/min and 28° C. for 48 h; a prepared ST15 fermentation broth was centrifuged at 10,000 r/min for 10 min, a supernatant was removed, the bacteria were collected and suspended in sterile water, a bacterial density was adjusted to about 1.0×10⁸ cfu/mL, and Tween 80 with a final concentration of 0.1% (v/v) was added; the above Bacillus altitudinis ST15 bacterial suspension was used to soak and treat rice seeds for 36 h at 25° C.-30° C., the rice seeds were taken out to be placed in a petri dish lined with filter paper, germination acceleration was conducted at 28° C. for 72 h in a moisturizing manner, and a germination rate, a shoot length and a root length were measured. Seed soaking in sterile water was used as a control, and 120 seeds were soaked in each treatment. The results showed that compared with the sterile water control, the ST15 bacterial suspension can significantly promote growth of rice roots (P<0.01) and growth of buds, but there was no difference in the seed germination rate between the two treatments, as shown in FIG. 8 and FIG. 9.

### Embodiment 6

Improvement of Drought Resistance of Rice Seedling by Bacillus altitudinis ST15

An ST15 seed broth was prepared according to the method of Embodiment 2, inoculated in a 250 mL conical flask containing 50 mL of a 50% NB liquid medium at a ratio of 1% (v/v), and cultured at 180 r/min and 28° C. for 48 h; a prepared ST15 fermentation broth was centrifuged at 10,000 r/min for 10 min, a supernatant was removed, the bacteria were collected and suspended in sterile water, a bacterial density was adjusted to about 1.0×10⁸ cfu/mL, and Tween 80 with a final concentration of 0.1% (v/v) was added; the Bacillus altitudinis ST15 bacterial suspension was used to soak and treat rice seeds for 36 h at 25° C.-30° C., the rice seeds were taken out to be placed in a petri dish lined with filter paper, germination acceleration was conducted at 28° C. for 48 h in a moisturizing manner, the treated rice seeds were placed at a room temperature for 120 h and a growth vigor of leaves and roots of rice seedlings was observed. The results showed that filter paper in the petri dish became relatively dry after being placed at room temperature for 120 h and the rice seedlings were dry. At this time, the seedlings germinated from the rice seeds treated with sterile water had few roots, weak buds, and no unfolded leaves, indicating that drought significantly restricted growth of the seedlings; and the seedlings germinated from the rice seeds treated with the ST15 bacterial suspension had more roots, were in a 1-leaf or 1-leaf 1-stem stage, and showed a strong drought resistance as shown in FIG. 10.

### Embodiment 7

Experiment of Bacillus altitudinis ST15 Bacterial Suspension on Controlling Bacterial Blight of Rice by Greenhouse

Xanthomonas oryzae pv. oryzae strain PX099 preserved in glycerol was taken out from an ultra-low temperature refrigerator at −70° C., inoculated on an NA solid plate by streaking and cultured at 28° C. for 48 h; a PX099 single colony was picked by using a sterilized toothpick to be inoculated into a 250 mL conical flask containing 50 mL of an NB liquid medium and cultured at 150 r/min and 28° C. for 48 h, a bacterial density was adjusted to about 1.0×10⁸ cfu/mL by using sterile water, Tween 80 at a final concentration of 0.1% (v/v) was added, and the pathogenic bacteria were used as a seed broth. An ST15 seed broth was prepared according to the method of Embodiment 2, inoculated in a 250 mL conical flask containing 50 mL of a 50% NB liquid medium at a ratio of 1% (v/v), and cultured at 180 r/min and 28° C. for 48 h; a prepared ST15 fermentation broth was centrifuged at 10,000 r/min for 10 min, a supernatant was removed, the bacteria were collected and suspended in sterile water, a bacterial density was adjusted to about 1.0×10⁸ cfu/mL, and Tween 80 with a final concentration of 0.1% (v/v) was added as a biocontrol bacteria suspension.

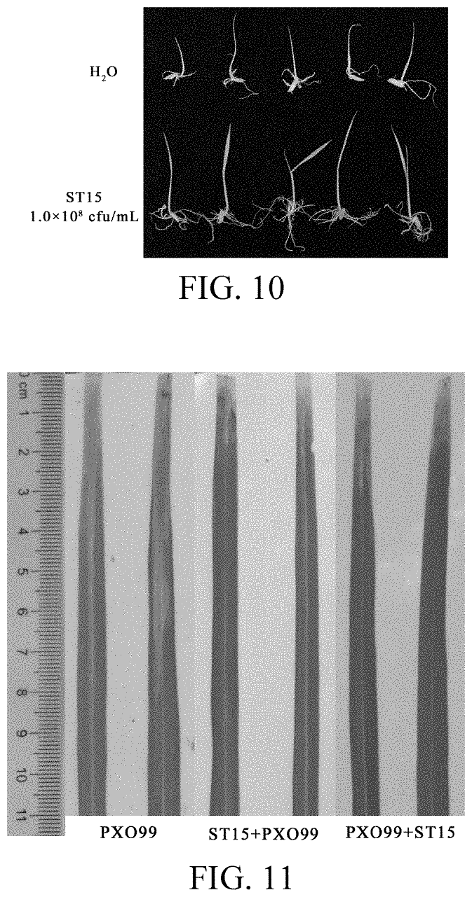

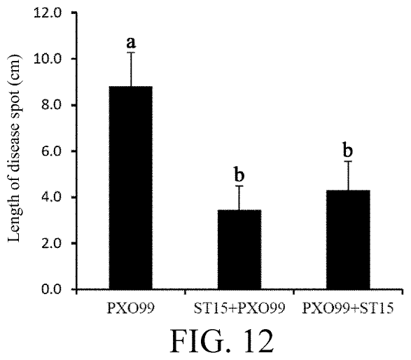

A susceptible rice variety IR24 at an end of a tillering stage was selected and 3 biocontrol experimental treatments were conducted: treatment 1, an ST15 biocontrol bacteria suspension of 1.0×10⁸ cfu/mL was sprayed, leaves were cut 24 h later, and the PX099 seed broth of the Xanthomonas oryzae pv. oryzae was inoculated and labeled as ST15+PX099; treatment 2, the leaves were cut first, the PX099 seed broth of the Xanthomonas oryzae pv. oryzae was inoculated, and the ST15 biocontrol bacteria suspension of 1.0×10⁸ cfu/mL was sprayed 24 h later and labeled as PX099+ST15; and treatment 3, leaves were cut and the PX099 seed broth of the Xanthomonas oryzae pv. oryzae was inoculated and labeled as PX099 as a control treatment. 10 rice plants were inoculated for each treatment, leaves of each plant were cut, 2 upper leaves of each plant were inoculated, and a length of disease spots was investigated 14 days after the inoculation. The results showed that compared with the control treatment only inoculated with the pathogen PX099, the lengths of the disease spots of the leaves subjected to the biocontrol treatments 1 and 2 were significantly reduced (both P<0.05) by 60.2% and 51.1%, respectively, but the difference was not significant between the two biocontrol treatments (both P>0.05) as shown in FIG. 11 and FIG. 12.

### Embodiment 8

Preparation of Bacillus altitudinis ST15 Biocontrol Inoculant and Effect Thereof on Controlling Bacterial Leaf Streak of Rice

The Bacillus altitudinis ST15 of the present invention stored in glycerol was taken, inoculated on an NA solid plate by streaking, and cultured at 28° C. for 36 h; an ST15 single colony was picked by using a sterilized toothpick to be inoculated into a 250 mL conical flask containing 50 mL of a 50% NB liquid medium and cultured at 180 r/min and 28° C. for 16 has a seed broth; and the seed broth was inoculated at a ratio of 2% (v/v) into a 1,000 mL conical flask containing 200 mL of the 50% NB liquid medium and cultured at 180 r/min and 28° C. for 48 h. 1.0% of fatty alcohol polyoxyethylene ether, 0.2% of xanthan gum, 0.3% of polydimethylsiloxane and 0.1% of sodium benzoate were added to the fermentation broth, a pH value was adjusted to about 4.5 by using dilute hydrochloric acid, and an ST15 biocontrol inoculant was obtained, aseptically sub-packaged and stored at a room temperature.

A use method of the biocontrol inoculant in controlling bacterial leaf streak of rice: the biocontrol inoculant was diluted by 40-60 times, spraying was conducted at an initial stage of the occurrence of the bacterial leaf streak of rice, and spraying was conducted 1 more time at an interval of 7-10 d and a good control effect can be achieved.

Application Cases:

In 2020, an ST15 biocontrol inoculant was used to conduct a field trial to control bacterial leaf streak of rice in the field to investigate a control effect on the bacterial leaf streak of rice at a test dose. The experiment was provided with 4 treatments of 10,000 mL of ST15 biocontrol inoculant/hectare, 15,000 mL of ST15 biocontrol inoculant/hectare, 1,500 mL of a 3% benziothiazolinone suspension/hectare, clean water as a control, and 600 kg water per hectare was sprayed evenly. The rice variety was C Liangyou 0861 and the disease was found in an investigation on August 7 (a booting stage of rice). A 1^{st} administration was conducted on August 10, a 2^{nd} administration was conducted on August 19, a disease index was investigated on the 11^{th} day after the 2^{nd} administration, and the control effect was subjected to a statistical analysis. The results were shown in Table 2 that on the 11^{th} day after the 2^{nd} administration, the control effect of the ST15 biocontrol inoculant at a dose of 15,000 mL/hectare was 63.8%, which was similar to the control effect of a chemical agent of the 3% benziothiazolinone suspension (66.2%); and the control effect of the ST15 biocontrol inoculant at a dose of 10,000 mL/hectare was 55.1%.

TABLE 2
Field trial results of ST15 biocontrol inoculant for
controlling bacterial leaf streak of rice

| | | Disease | Disease | |
|---|---|---|---|---|
| Treatment | | index | index | |
| | Dose | admin- | after | Control |
| | (mL/ | istration | admin- | effect |
| Agent | hectare) | before | istration | (%) |
| ST15 biocontrol | 10,000 | 1.31 | 8.62 | 55.1 b |
| inoculant | | | | |
| ST15 biocontrol | 15,000 | 1.18 | 6.26 | 63.8 ab |
| inoculant | | | | |
| 3% benziothiazolinone | 1,500 | 1.07 | 5.31 | 66.2 a |
| suspension | | | | |

| | | | |
|---|---|---|---|
| Clean water as control | 1.25 | 18.33 | / |

In the above embodiments, the Bacillus altitudinis strain ST15 of the present invention has a relatively high antagonistic activity to Xanthomonas oryzae pv. oryzae and Xanthomonas oryzae pv. oryzicola, promotes growth and improves a drought resistance of rice, has a relatively strong tolerance to 3 main chemical microbicides for controlling bacterial diseases of rice, has a better control effect on bacterial blight of rice and bacterial leaf streak of rice, can replace or reduce chemical pesticides, improves safety of food and ecological environment, and has a good development and use prospect.

SEQUENCE LISTING

SEQ ID NO. l:
Jiangsu Academy of Agricultural Sciences
Bacillus spp. antagonizing Xanthomonas oryzae and use thereof
A gyrB gene sequence of a Bacillus altitudinis ST15

| | |
|---|---|
| 1 | tacggtgcgtgctagtcgacggcagcggttataaagtatctggcggtctgcatggtgtag |
| 61 | gggcatctgttgttaatgcgttatctacgaccttagacgtgaccgtataccgtgatggaa |
| 121 | aaattcattaccagcaattcaaacgcggcgttccagttggagatttagaggtcattggtg |
| 181 | aaacagatgtaacagggacaacgactcattttgtgccagatccagaaattttcactgaaa |
| 241 | ccattgaatttgattacgacacacttgctaaccgtgtacgtgagttagctttcttaacaa |
| 301 | aaggcgtcaacatcatcattgaagacttacgtgaaggcaaagagcgaagaaacgaatact |
| 361 | gctatgaaggcggtattaagagctatgtagaacatttgaatcgctcgaaggaagtcgttc |
| 421 | atgaagaaccagtttacatcgagggtgaaaaagacggaatcaccgttgaagtggcactgc |
| 481 | aatacaacgattcctatacaagcaatatttattctttcgccaacaacatcaacacatatg |
| 541 | aaggcggaacacacgaagctggctttaaaaccggtctgacgcgtgtcatcaatgattatg |
| 601 | ctcgtaaaaatggcgtattcaaagatggagactcgaatttgagcggtgaagatgtacgag |
| 661 | aaggcttaacagccattatctctatcaaacatccagaccctcaattcgaaggacaaacga |
| 721 | agacaaagctcggtaactcagaagcaagaaccattaccgactccctcttctccgaagcac |
| 781 | ttgagaaattcctcttagagaaccctgatgctgcaaagaaaattgtggagaaaggtgtga |
| 841 | tggcagctcgtgcaagaatggcctgccaaaaaaggcacgtgagctgacaagacgtaaaag |
| 901 | tgcactggaagtctctagcttgcctgcaaa |

1. A method comprising performing fermentation with Bacillus altitudinis ST15, having the deposit number of CGMCC No. 20156, in a fermentation medium.

2. The method according to claim 1, wherein a fermentation product is obtained.

3. The method according to claim 1, wherein a fermentation broth is obtained and optionally the fermentation broth is centrifuged and filtered to obtain a sterile fermentation filtrate.

4. The method according to claim 3, further comprising resuspending the Bacillus altitudinis ST15 from the fermentation broth to obtain a bacterial suspension containing the Bacillus altitudinis ST15.

5. The method according to claim 3, further comprising adding auxiliary agents to the fermentation broth to obtain a biocontrol inoculant containing the Bacillus altitudinis ST15.

6. A method comprising applying Bacillus altitudinis ST15 having the deposit number of CGMCC No. 20156, a fermentation product containing the Bacillus altitudinis ST15, a fermentation broth containing the Bacillus altitudinis ST15 or a sterile fermentation filtrate obtained therefrom, or a bacterial suspension containing the Bacillus altitudinis ST15 to rice or a rice seedling so as to promote growth of the rice or/and improve a drought resistance of the rice seedling.

7. The method according to claim 6, wherein the Bacillus altitudinis ST15, the fermentation product, the fermentation broth or the sterile fermentation filtrate, or the bacterial suspension is used to treat a rice seed for 36 h-48 h at 25° C.-30° C.

8. The method according to claim 7, wherein the rice seed is treated by soaking.

9. A method comprising applying Bacillus altitudinis ST15 having the deposit number of CGMCC No. 20156, a fermentation product containing the Bacillus altitudinis ST15, a fermentation broth containing the Bacillus altitudinis ST15 or a sterile fermentation filtrate obtained therefrom, or a bacterial suspension containing the Bacillus altitudinis ST15 to rice so as to control bacterial blight of the rice and/or bacterial leaf streak of the rice.

10. The method according to claim 9, wherein controlling the bacterial blight of rice is as follows: spraying before rice is infected with Xanthomonas oryzae pv. oryzae or after rice is infected with Xanthomonas oryzae pv. oryzae; controlling the bacterial leaf streak of rice is as follows: spraying before rice is infected with Xanthomonas oryzae pv. oryzicola or at an initial stage of the infection and spraying 1-2 times at an interval of 7-10 d.
